(19) United States
(12) Patent Application Publication
(10) Pub. No.: US 2025/0180148 A1
(43) Pub. Date: Jun. 5, 2025

(54) INSTRUMENT TEE WITH REDUCED OR ELIMINATED DEAD ZONES

(71) Applicant: Contour Biosolutions, LLC, Middlesex, NJ (US)

(72) Inventor: Louis Bongiorno, Millington, NJ (US)

(73) Assignee: Contour Biosolutions, LLC, Middlesex, NJ (US)

(21) Appl. No.: 18/961,933

(22) Filed: Nov. 27, 2024

Related U.S. Application Data
(60) Provisional application No. 63/605,630, filed on Dec. 4, 2023.

(51) Int. Cl. F16L 41/02; F16L 41/00; F16L 55/00

(52) U.S. Cl. CPC F16L 41/021; F16L 41/008; F16L 55/00

(57) ABSTRACT

An instrument tee includes a main fluid passageway, a pressure fluid passageway, at least a first clamp member, and a gasket. The main fluid passageway includes a first fluid port, a second fluid port, and a pressure fluid passageway between the first fluid port and the second fluid port. The pressure fluid passageway includes a proximal end fluidly connected to the pressure fluid passageway. The first clamp member is configured to connect an instrument to a distal end of the pressure fluid passageway. The gasket is configured to connect between at least a portion of a top surface of the pressure fluid passageway and a bottom surface of the instrument when the instrument is connected to the instrument tee by the first clamp member. A first inner diameter at a proximal end of the pressure fluid passageway is not the same as a second inner diameter at a distal end of the pressure fluid passageway.

### CROSS REFERENCES AND PRIORITIES

This application claims priority from U.S. Provisional Application No. 63/605,630 filed on 4 Dec. 2023, the teachings of which are incorporated by reference herein in their entirety.

### BACKGROUND

Various devices exist for measuring conditions—such as pressure and other relevant flow characteristics of fluids—within disposable tubing. One prior art device for measuring pressure within tubing utilizes disposable pressure sensors with a luer lock fitting, as in U.S. Pat. No. 4,576,181. However, this device can only be used with small tubing sizes and their associated low flow rates. Further, this device requires a dedicated electronic interface and display for the pressure readout. In general, disposable pressure sensors that are provided pre-sterilized have limitations in that they cannot be calibrated before their use. Additionally, this device fails to function with analog instruments that are often used in various chemical manufacturing processes.

Another prior art device is a mini sanitary gauge tee made of stainless steel with hose barb adapters. Although this device can measure pressure within tubing, including by way of analog instruments, it's open design results in constant contact with the diaphragm of the stainless steel gauge. As a result, contamination of the gauge can occur. Furthermore, considerable hold-up volume in the range of 5 to 50 mL can result.

Especially when operating with biologics or pharmaceuticals, minimal hold-up volume is preferred. Hold-up is an amount of fluid in a system that remains stagnant, generally caused by a branch or tee in a line for a valve, gauge, or measuring line. If allowed to remain in place, such hold-up fluid can dilute the end product being produced on a line with a gauge. Furthermore, it is possible to entrap air in deadlegs, which can pose problems for the manufacturing process and equipment.

Anderson Instrument Co., Inc. of Mississauga, Ontario, Canada describes a zero dead-leg instrument fitting that can be installed on lines as small as half an inch in diameter. The fitting is a tee and can be connected to a gauge or transmitter. The fitting is made of stainless steel, thus the liquid flow is still in contact with stainless steel. The fitting is intended for multiple uses.

U.S. Pat. No. 5,000,049 to Cooper et al. discloses an apparatus for measuring fluid pressure in a tube of a catheter. The tube can include threads for receiving a gauge. The tube is made of a biocompatible, nonpyrogenic and sterilizable material. A sealing diaphragm is provided between the fluid path and the pressure-sensitive diaphragm, preferably made of polyethylene, polyurethane, polyvinylchloride, or the like. Thus, the diaphragm sends a read-out to a gauge pointer and there is no contact of the fluid with metal. However, such a device could not be used readily in a biopharmaceutical process, as the gauge must be calibrated before and after use. Calibrating the gauge after use may jeopardize the next batch processed if the calibration fails.

U.S. Pat. No. 6,117,086 to Shulze discloses a physiological pressure transducer coupled to a catheter through a disposable dome containing a shaped, compliant isolation media, which is in intimate contact with the reusable transducer diaphragm. The disposable dome is in the shape of a tee. The isolation media preferably forms inside of all sharp corners around the outer diameter of the diaphragm where it contacts the disposable dome, thus providing an extremely smooth and essentially straight fluid path without the entrapment of bubbles. Again, this device requires a gauge that must be calibrated before and after use, and thus could not be used in biopharmaceutical processes.

U.S. Pat. No. 6,978,636 to Motush et al. discloses a portable device for measuring refrigerant pressure, including a hose with a first end connected to an actuator on a pressurized container and a second end connectable to a service port of an automobile air conditioner. A T-connector with a check valve and a pressure gauge is disposed in the hose. In the preferred embodiment, at least one barb of the T-connector is rotatable and is a separate piece from the body of the T-connector. This enable the T-connector to rotate with respect to the hose so that the pressure gauge will always be viewable by the user. The T-connector is preferably made of die-cast zinc, thus there is metal contact with the fluid. The device interfaces with a threaded pressure gauge and allows fluid to pass up into the body of the gauge. Such a device could not be used in biopharmaceutical processes because the fluid is in contact with the gauge itself, allowing for contamination.

U.S. Pat. No. 7,373,825 to Fennington, Jr. discloses a disposable sanitary gauge tee with no dead zone for use in pressure measurement of fluid in disposable tubing including a body having a main fluid passageway in fluid connection with a proximal end of a pressure fluid passageway. The main fluid passageway including a fluid port and a second fluid port. The pressure fluid passageway including a distal end for removable attachment of a sanitary pressure gauge and a biocompatible gauge protector for isolation of the fluid from the sanitary pressure gauge. The device includes a long, blunt edged pressure fluid passageway which can result in inaccurate pressure readings at the pressure gauge relative to the pressure in the main fluid passageway as well as sheering or impact buildup of solid particles flowing through the main fluid passageway past the pressure fluid passageway.

The need exists, therefore, for an improved device for measuring conditions—such as pressure and other relevant flow characteristics of fluids—within disposable tubing which reduces or eliminates contamination risks from metal parts, accurately measures parameters such as pressure within the tubing, reduces or eliminates dead zones, and maintains a closed flow path even when not connected to the measuring instrument.

### SUMMARY

Described herein is an instrument tee comprising a main fluid passageway, a pressure fluid passageway, a first clamp member, and a gasket. The main fluid passageway comprises a first fluid port, a second fluid port opposite the first fluid port, and a pressure fluid passageway port between the first fluid port and the second fluid port. The pressure fluid passageway comprises a proximal end fluidly connected to the pressure fluid passageway port and having a first inner diameter and a first outer diameter. The pressure fluid passageway also comprises a distal end having a second inner diameter and a second outer diameter. The first inner diameter is not the same as the second inner diameter. The first clamp member is configured to connect an instrument to the distal end of the pressure fluid passageway. The gasket includes a first surface which is configured to connect to at least a portion of a top surface of the pressure fluid passageway, and a second surface which is configured to connect to a bottom surface of the instrument when the instrument is connected to the instrument tee by the first clamp member.

In some embodiments, the first inner diameter may be greater than the second inner diameter. In other embodiments, the first inner diameter may be less than the second inner diameter.

In certain embodiments, the first fluid port may comprise a first hose barb. In some embodiments, the second fluid port may comprise a second hose barb.

The instrument tee may comprise an instrument removably connected to the pressure fluid passageway by at least the first clamp member. In some embodiments, the instrument may be a pressure gauge. In certain such embodiments, the first clamp member may be integrally connected to an end of the pressure gauge. In other embodiments, the instrument may be a centrifugal pump. In certain such embodiments, the first clamp member may be integrally connected to an end of the centrifugal pump. In still other embodiments, the instrument may be a valve. In certain such embodiments, the first clamp member may be integrally connected to an end of the valve. In still other embodiments, the instrument may be a positive displacement pump. In certain such embodiments, the first clamp member may be integrally connected to an end of the positive displacement pump. The gasket may be configured to fluidly isolate the pressure fluid passageway from the instrument.

In some embodiments, the first clamp member may comprise a first clamp member first section and a first clamp member second section. The first clamp member first section may have a first semi-circular wall, a first wall first engagement member, and a first wall second engagement member. The first semi-circular wall may have a first inner surface, a first outer surface, a first wall first end point, and a first wall second end point. The first wall first engagement member may be attached to the first wall outer surface at the first wall first end point and may comprise a first hinge section. The first wall second engagement member may be attached to the first outer surface at the first wall second end point and may comprise a first plurality of inwardly facing teeth. The first clamp member second section may have a second semi-circular wall, a second wall first engagement member, and a second wall second engagement member. The second semi-circular wall may have a second inner surface, a second outer surface, a second wall first end point, and a second wall second end point. The second wall first engagement member may be attached to the second outer surface at the second wall first end point and may comprise a second hinge section. The second wall second engagement member may be attached to the second outer surface at the second wall second end point and may comprise a first plurality of outwardly facing teeth. The first clamp member first section may be configured to connect to the first clamp member second section by mating the first hinge section to the second hinge section and mating the first plurality of inwardly facing teeth to the first plurality of outwardly facing teeth.

In certain embodiments, the instrument tee may further comprise a force plate. The force plate may have a force plate top surface, a portion of which may be configured to interact with the first clamp member. The force plate may also have a force plate bottom surface which may be configured to interact with at least a portion of the top surface of the pressure fluid passage. The force plate may also have a force plate lip extending from the force plate bottom surface which may be configured to interact with a top surface of the instrument when the instrument is connected to the instrument tee by the first clamp member.

In some embodiments, the instrument tee may further comprise a second clamp member configured to connect the instrument to the distal end of the pressure fluid passageway. In certain such embodiments, the second clamp member may be integrally connected to the distal end of the pressure fluid passageway.

The second clamp member, when present, may comprise a second clamp member first section and a second clamp member second section. The second clamp member first section may have a third semi-circular wall, a third wall first engagement member, and a third wall second engagement member. The third semi-circular wall may have a third inner surface, a third outer surface, a third wall first end point, and a third wall second end point. The third wall first engagement member may be attached to the third outer surface at the third wall first end point. The third wall second engagement member may be attached to the third outer surface at the third wall second end point. The second clamp member second section may have a fourth semi-circular wall, a fourth wall first engagement member, and a fourth wall second engagement member. The fourth semi-circular wall may have a fourth inner surface, a fourth outer surface, a fourth wall first end point, and a fourth wall second end point. The fourth wall first engagement member may be attached to the fourth outer surface at the fourth wall first end point. The fourth wall second engagement member may be attached to the fourth outer surface at the fourth wall second end point. The third wall first engagement member may be configured to connect to the fourth wall first engagement member. The third wall second engagement member may be configured to connect to the fourth wall second engagement member.

### BRIEF DESCRIPTION OF FIGURES

FIG. 1 is a perspective view of an instrument tee with an attached instrument.

FIG. 2 is a perspective view of a main fluid passageway.

FIG. 3 is a cross section view of a main fluid passageway.

FIG. 4A and FIG. 4B are exploded perspective views of an embodiment of an instrument tee.

FIG. 5A and FIG. 5B are assembled perspective views of the embodiment of an instrument tee of FIG. 4A and FIG. 4B.

FIG. 6 is a cross section view of the embodiment of an instrument tee of FIG. 4A and FIG. 4B.

FIG. 7A and FIG. 7B are exploded perspective views of an alternative embodiment of an instrument tee.

FIG. 8A and FIG. 8B are assembled perspective views of the alternative embodiment of an instrument tee of FIG. 7A and FIG. 7B.

FIG. 9 is a cross section view of the alternative embodiment of an instrument tee of FIG. 7A and FIG. 7B.

FIG. 10A and FIG. 10B are exploded perspective views of a second alternative embodiment of an instrument tee.

FIG. 11A and FIG. 11B are assembled perspective views of the second alternative embodiment of an instrument tee of FIG. 10A and FIG. 10B.

FIG. 12 is a cross section view of the second alternative embodiment of an instrument tee of FIG. 10A and FIG. 10B.

FIG. 13 is a perspective view of an embodiment of an instrument tee connected to a centrifugal pump.

FIG. 14 is a cross section view of the instrument tee connected to a centrifugal pump of FIG. 13.

FIG. 15 is a perspective view of an embodiment of an instrument tee connected to a valve.

FIG. 16 is a cross section view of the instrument tee connected to a valve of FIG. 15.

FIG. 17 is a perspective view of an embodiment of an instrument tee connected to a positive displacement pump.

FIG. 18 is a cross section view of the instrument tee connected to a positive displacement pump of FIG. 17.

### DETAILED DESCRIPTION

Disclosed herein are instrument tees. The instrument tees are described below with reference to the Figures. As described herein and, the following numbers refer to the following structures as noted in the Figures.
10 refers to an instrument tee.
20 refers to an instrument.
21 refers to a centrifugal pump.
22 refers to a valve.
23 refers to a positive displacement pump.
100 refers to a main fluid passageway.
110 refers to a first fluid port.
111 refers to a first hose barb.
120 refers to a second fluid port.
121 refers to a second hose barb.
130 refers to a pressure fluid passageway port.
200 refers to pressure fluid passageway.
210 refers to proximal end.
211 refers to a first inner diameter.
220 refers to a distal end.
221 refers to a second inner diameter.
222 refers to a second outer diameter.
230 refers to a top surface.
300 refers to a first clamp member.
310 refers to a first clamp member first section.
311 refers to a first semi-circular wall.
312 refers to a first inner surface.
313 refers to a first outer surface.
314 refers to a first wall first end point.
315 refers to a first wall second end point.
316 refers to a first wall first engagement member.
317 refers to a first hinge section.
318 refers to a first wall second engagement member.
319 refers to inwardly facing teeth.
320 refers to a first clamp member second section.
321 refers to a second semi-circular wall.
322 refers to a second inner surface.
323 refers to a second outer surface.
324 refers to a second wall first end point.
325 refers to a second wall second end point.
326 refers to a second wall first engagement member.
327 refers to a second hinge section.
328 refers to a second wall second engagement member.
329 refers to outwardly facing teeth.
330 refers to a first joining member.
331 refers to a hinge mount.
332 refers to joining member teeth.
340 refers to a thumb protrusion.
400 refers to a gasket.
410 refers to a first surface.
420 refers to a second surface.
500 refers to a force plate.
510 refers to a force plate top surface.
520 refers to a force plate bottom surface.
530 refers to a force plate lip.
600 refers to a second clamp member.
610 refers to a second clamp member first section.
611 refers to a third semi-circular wall.
612 refers to a third inner surface.
613 refers to a third outer surface.
614 refers to a third wall first end point.
615 refers to a third wall second end point.
616 refers to a third wall first engagement member.
617 refers to a third wall second engagement member.
620 refers to a second clamp member second section.
621 refers to a fourth semi-circular wall.
622 refers to a fourth inner surface.
623 refers to a fourth outer surface.
624 refers to a fourth wall first end point.
625 refers to a fourth wall second end point.
626 refers to a fourth wall first engagement member.
627 refers to a fourth wall second engagement member.

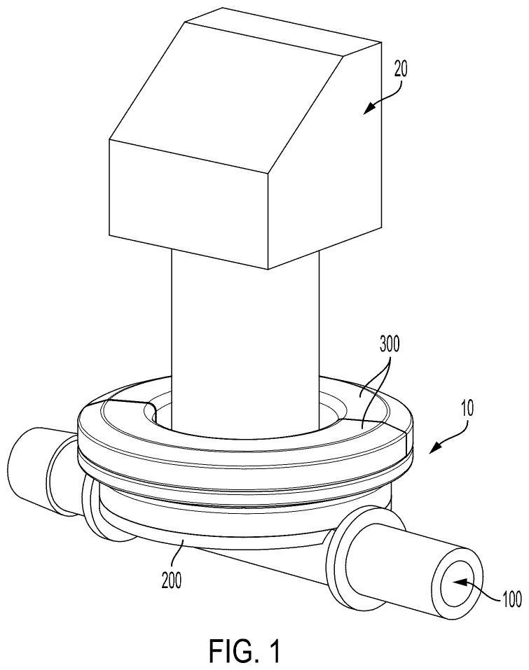

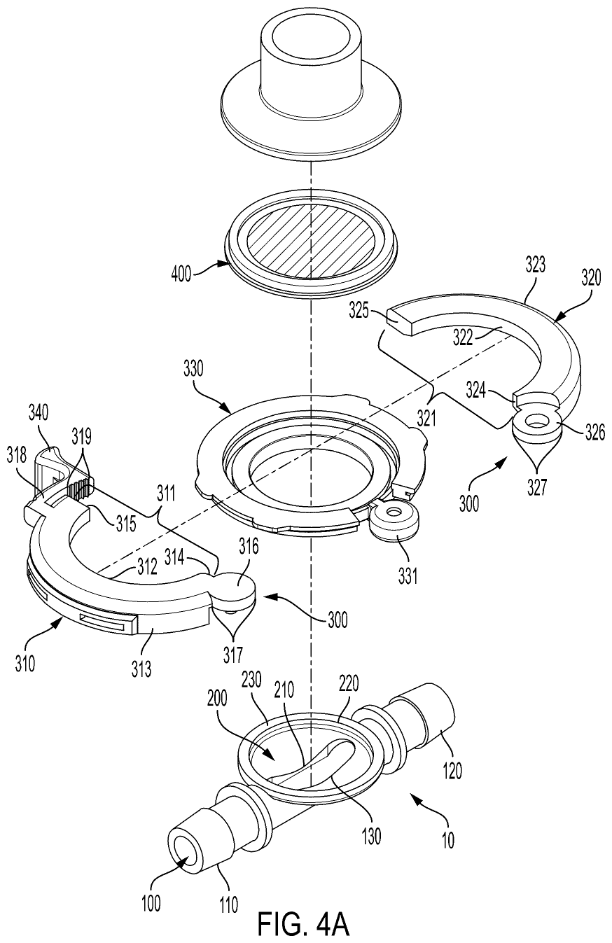

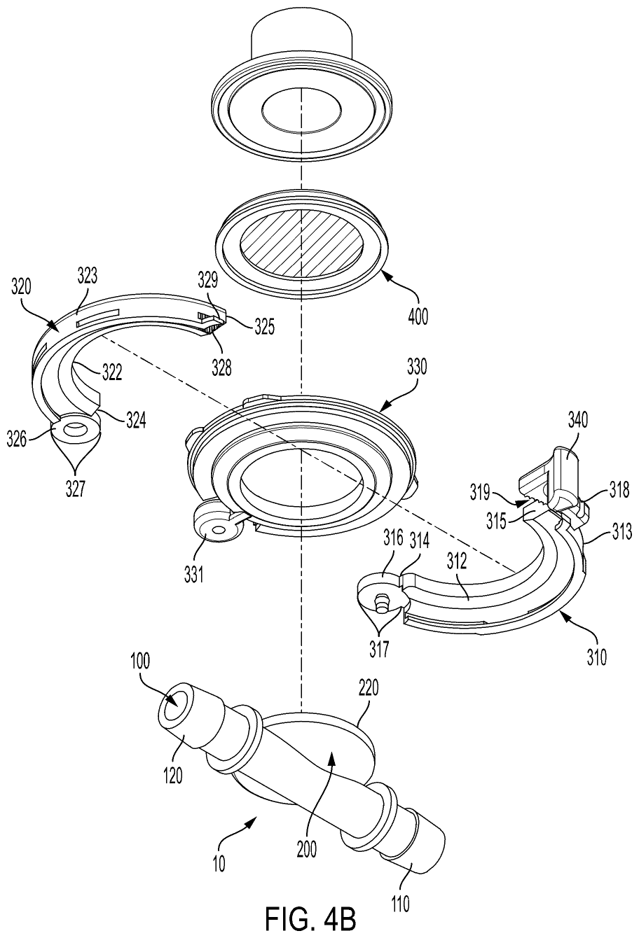

FIG. 1 shows an assembled version of an instrument tee (10) with an instrument (20) connected thereto. As shown in FIG. 1, the instrument tee comprises a main fluid passageway (100), a pressure fluid passageway (200), a first clamp member (300), and a gasket (400 as shown in FIGS. 4A, 4B, 6, 7A, 7B, 9, 10A, 10B, and 12). While FIG. 1 shows the instrument being an electronic pressure sensor, one of ordinary skill will recognize that the instrument may be any number of electronic or analog sensors.

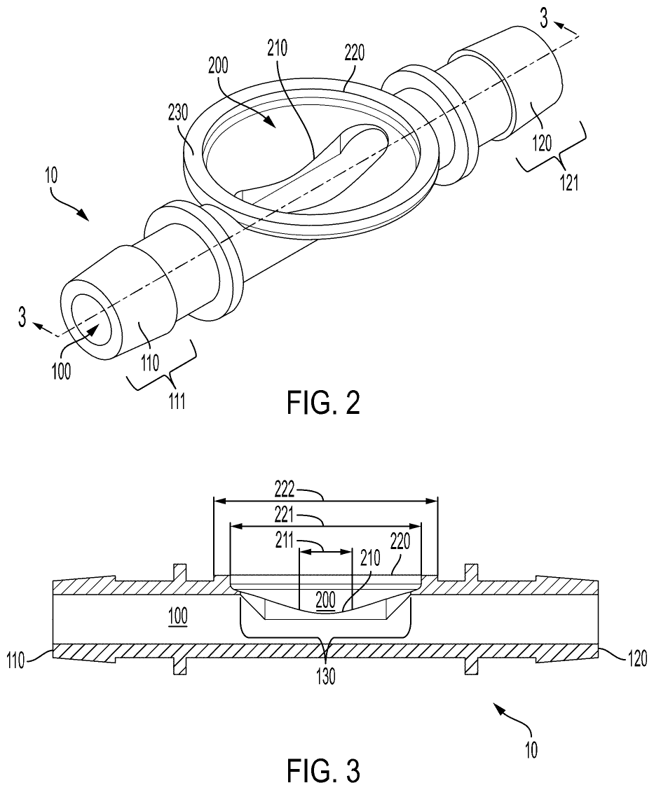

FIG. 2 shows a perspective view of an instrument tee (100) without an instrument (20 as shown in FIG. 1) connected thereto. As shown in FIG. 2, the main fluid passageway (100) of the instrument tee comprises a first fluid port (110) and a second fluid port (120) opposite the first fluid port. The opposing fluid ports are configured to permit connection of conduits—non-limiting examples of which include a hose, a tube, a line, or the like—of a chemical manufacturing system. In some embodiments, such as shown in FIG. 2, the connection to the first fluid port may involve a first hose barb (111) which provides a frictional engagement between the conduit and the first fluid port when the conduit is advanced over and around the first fluid port. Similarly, the connection to the second fluid port may involve a second hose barb (121) which provides a frictional engagement between the conduit and the second fluid port when the conduit is advanced over the second fluid port. One non-limiting alternative to a hose barb is a threaded fluid port to which a threaded fitting at the end of a conduit may be attached.

Further shown in FIG. 2 is the pressure fluid passageway (200). As shown in FIG. 2, the pressure fluid passageway includes a proximal end (210) and a distal end (220) with the proximal end attached to the main fluid passageway (100). Preferably, the connection between the proximal end and the main fluid passageway will be an integral connection—such as achieved by manufacturing the main fluid passageway and the pressure fluid passageway of a single integral piece of material. However, in some embodiments, the pressure fluid passageway may be removably connected to the main fluid passageway such as by threading the proximal end of the pressure fluid passageway into or onto a pressure fluid passageway port (130 as shown in FIG. 3) of the main fluid passageway.

The instrument tee (10) is shown in cross section in FIG. 3. As shown in FIG. 3, the main fluid passageway (100) includes a pressure fluid passageway port (130) between the first fluid port (110) and the second fluid port (120). The proximal end (210) of the pressure fluid passageway (200) is connected—integrally or removably as described herein—to the pressure fluid passageway port.

The proximal end (210) of the pressure fluid passageway (200) has a first inner diameter (211). The distal end (220) of the pressure fluid passageway has both a second inner diameter (221) and a second outer diameter (222). As shown in FIG. 3, the first inner diameter is not the same as the second inner diameter. In some embodiments, the first inner diameter may be greater than the second inner diameter such that the inner diameter of the pressure fluid passageway necks down as fluid advances from the proximal end to the distal end as shown in FIG. 3. For example, a ratio between the first inner diameter and the second inner diameter may be in a range selected from the group consisting of between 1.1:1 and 10:1, between 1.1:1 and 7.5:1, between 1.1:1 and 5:1, between 1.1:1 and 2.5:1, between 1.5:1 and 10:1, between 1.5:1 and 7.5:1, between 1.1:1 and 5:1, between 1.1:1 and 2.5:1, between 2:1 and 10:1, between 2:1 and 7.5:1, and between 2:1 and 5:1. However, one of ordinary skill can easily envision how, in other embodiments, the first inner diameter may be less than the second inner diameter such that the inner diameter of the pressure fluid passageway necks up as fluid advances from the proximal end to the distal end. For example, a ratio between the first inner diameter and the second inner diameter may be in a range selected from the group consisting of between 1:1.1 and 1:10, between 1:1.1 and 1:7.5, between 1:1.1 and 1:5, between 1:1.1 and 1:2.5, between 1:1.5 and 1:10, between 1:1.5 and 1:7.5, between 1:1.5 and 1:5, between 1:1.5 and 1:2.5, between 1:2 and 1:10, between 1:2 and 1:7.5, and between 1:2 and 1:5.

While FIG. 3 shows the pressure fluid passageway having a circular profile—and thus having inner and outer diameters (211, 221, 222)—other embodiments may exist. For example, the pressure fluid passageway may have a polygonal profile in which case the pressure fluid passageway will have a first area in a plane parallel to the main fluid passageway (100) at the proximal end (210) and a second area in a plane parallel to the main fluid passageway at the distal end (220). In some embodiments, the first area may be greater than the second area while in other embodiments the first area may be less than the second area.

The first clamp member (300) is configured to connect an instrument (20 as shown in FIG. 1) to the distal end (220) of the pressure fluid passageway (200) as shown in FIG. 4A and FIG. 4B. The first clamp member may come in many forms, one of which is shown in FIG. 4A and FIG. 4B in which the first clamp member comprises two section, a first clamp member first section (310) and a first clamp member second section (320).

As shown in FIG. 4A and FIG. 4B, which are exploded side views, the first clamp member first section (310) may have a first semi-circular wall (311). Attached to opposing end points (314 and 315) of the first semi-circular wall are a first wall first engagement member (316) and a first wall second engagement member (318). Similarly, the first clamp member second section (320) includes a second semi-circular wall (321). Attached to opposing end points (324 and 325) of the second semi-circular wall are a second wall first engagement member (326) and a second wall second engagement member (328).

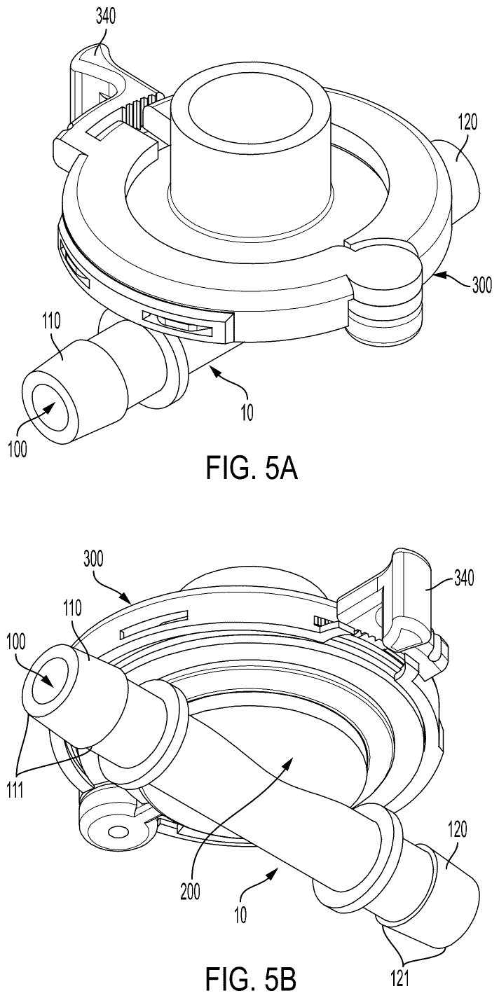

In the embodiment shown in FIG. 4A and FIG. 4B, the first wall first engagement member (316) may comprise a first hinge section (317) while the second wall first engagement member (326) may comprise a second hinge section (327). When assembled as shown in FIG. 5A and FIG. 5B, the first clamp member first section is configured to connect (partially) to the first clamp member second section by mating the first hinge section to the second hinge section.

Also shown in the embodiment in FIG. 4A and FIG. 4B, the first wall second engagement member (318) comprises a first plurality of inwardly facing teeth (319) while the second wall second engagement member (328) comprises a first plurality of outwardly facing teeth (329). When assembled as shown in FIG. 5A and FIG. 5B, the first clamp member first section is configured to connect (partially) to the first clamp member second section by mating the first plurality of inwardly facing teeth to the first plurality of outwardly facing teeth. In conjunction with mating the first hinge section (317) to the second hinge section (327), mating the first plurality of inwardly facing teeth to the first plurality of outwardly facing teeth provides for an assembled first clamp (300).

In some embodiments, the first wall second engagement member (318) and/or the second wall second engagement member (328) may include a thumb protrusion (340). When present, the thumb protrusion(s) allow a user to easily pivot the first clamp member first section (310) and the first clamp member second section (320) about an axis formed by the first hinge section (317) and the second hinge section (327) to mate the first plurality of inwardly facing teeth (319) with the first plurality of outwardly facing teeth (329) during assembly of the first clamp (300).

In some embodiments, portions of the first clamp member (300)—including one or both of the first clamp member first section (310) and/or the first clamp member second section (320)—may be integrally connected to the pressure fluid passageway (200) at the distal end (220) thereof. Such integral connection may be formed by manufacturing the pressure fluid passageway and the respective section(s) of the first clamp member of a single integral piece of material.

Alternatively, the first clamp member (300)—including one or both of the first clamp member first section (310) and/or the first clamp member second section (320)—may be removably connected to the pressure fluid passageway (200) at the distal end (220) thereof. In such embodiments, the distal end may include a flange. The first semi-circular wall (311) may include a first clamping surface which is angled inwardly from the first inner surface (312) towards the first outer surface (313). Similarly, the second semi-circular wall (321) may include a second clamping surface which is angled inwardly from the second inner surface (322) towards the second outer surface (323). As the first clamp is assembled about the distal end of the pressure fluid passageway, the inwardly angled clamping surfaces force the flanged end of the pressure fluid passageway and a corresponding flange on the instrument (20) closer to one another to achieve a tight, aseptic sealing between the two.

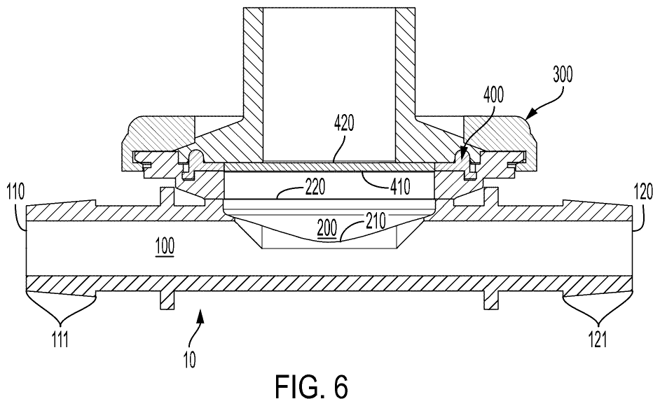

FIG. 4A and FIG. 4B also show a gasket (400), further details of which are shown in FIG. 6 which is a cross-section view of the embodiment shown in FIG. 4A and FIG. 4B. As shown in FIG. 6, the gasket includes a first surface (410) and a second surface (420) opposite the first surface. When assembled with the first clamp member (300) connecting an instrument (20) to the instrument tee (10), the first surface is configured to connect to at least a portion of a top surface (230) of the pressure fluid passageway (200) while the second surface is configured to connect to a bottom surface of the instrument. In some embodiments, the gasket may be configured to fluidly isolate the pressure fluid passageway from the instrument by blocking the flow of fluid through the distal end (220) of the pressure fluid passageway. In some embodiments, hermetic sealing may be formed by the gasket by having an integral membrane across the contact surface of the gasket which isolates the components from atmosphere. An instrument may be placed on the second surface—which is the external surface of the integral membrane portion of the gasket—to provide the interfaced from the instrument to access readings from outside of the aseptic process lumen such that expensive instrumentation may remain in-place while the sterilized single-use process components may be replaced. The integral membrane may be connected to the gasket in a number of manners. In some embodiments, the integral membrane may be welded to the body of the gaskets. In other embodiments, a mechanical interface created by a series of interlocking lips on the circumferential outer edge of the integral membrane and the circumferential inner edge of the gasket may connect the integral membrane to the gasket.

The gasket (400) may be fabricated of any number of materials utilizing any number of manufacturing techniques. Non-limiting examples of such materials include natural and synthetic rubbers. Non-limiting examples of such manufacturing techniques include injection molding and additive manufacturing (also referred to as 3D printing).

In some embodiments, the first clamp member (300) may include a first joining member (330) as shown in FIG. 4A and FIG. 4B. When present, the first joining member may include a cylindrical section having a number of features—such as a hinge mount (331) which assist in attaching the first hinge section to the second hinge section—connected thereto to assist in securely connecting the first clamp member first section (310) to the first clamp member second section (320).

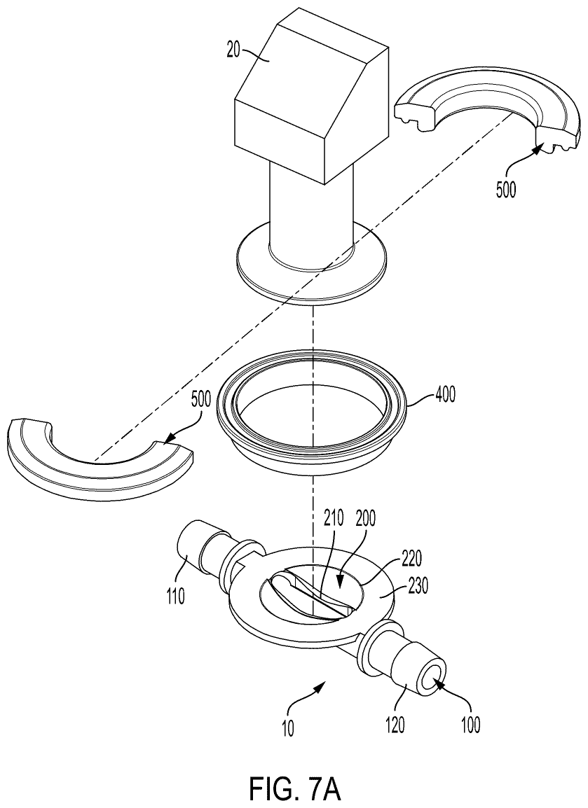

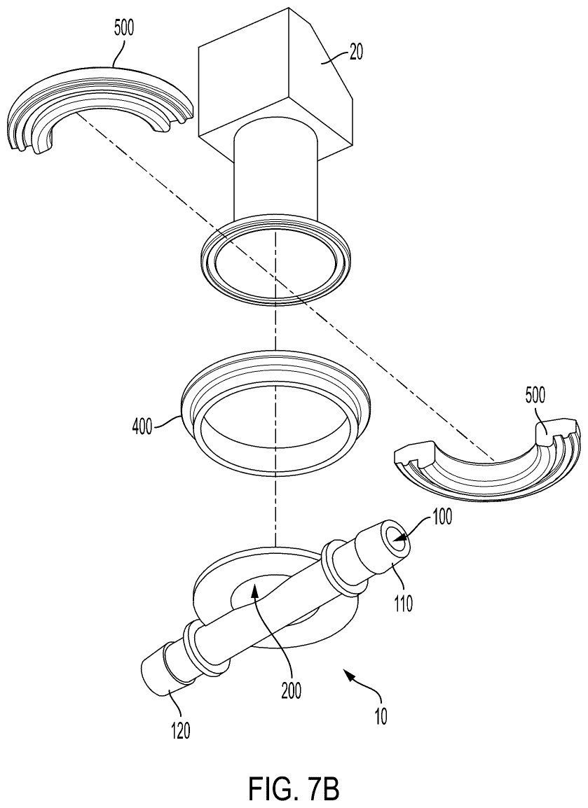

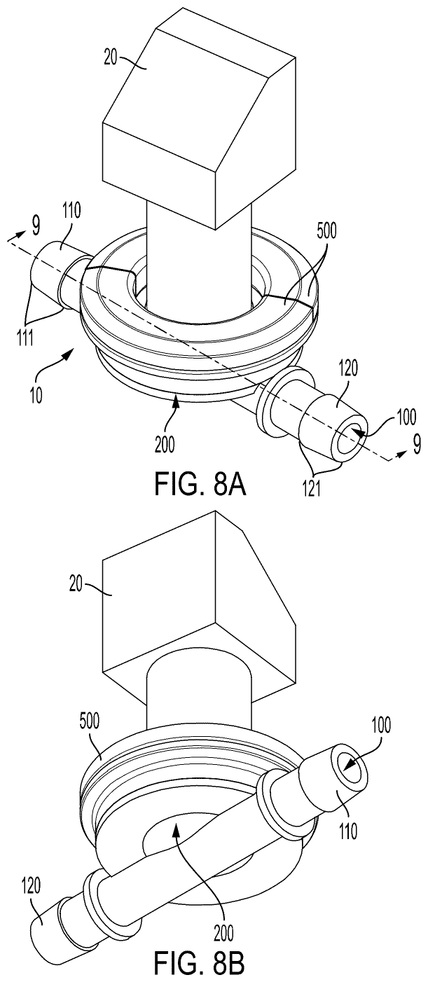

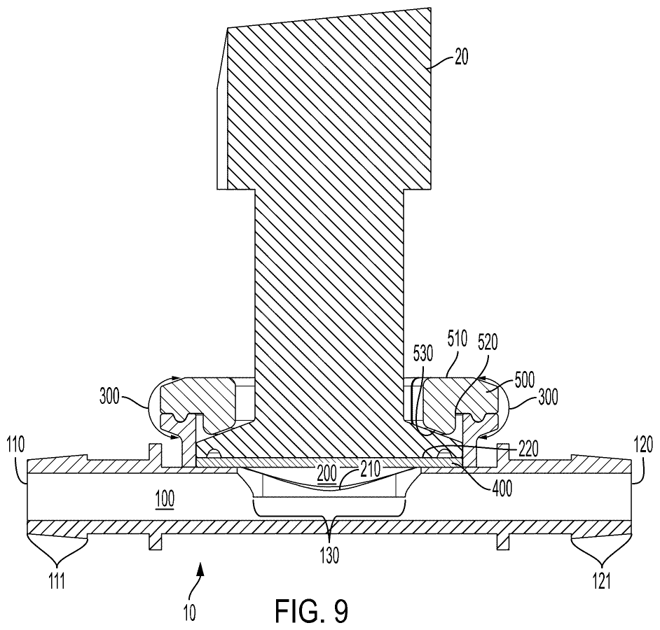

FIG. 7A through FIG. 9 show an alternative embodiment in which the instrument tee (10) includes a force plate (500) with FIG. 7A and FIG. 7B showing an exploded view thereof, FIG. 8A and FIG. 8B showing an assembled view thereof, and FIG. 9 showing the embodiment in cross section. As shown in the Figures, when the force plate is present, it may include a force plate top surface (510) and a force plate bottom surface (520). In assembled form, at least a portion of the force plate top surface is configured to interact with the first clamp member (300) such that, as the first clamp member is tightened around the instrument (20) and the pressure fluid passageway (200), the first clamp member advances the force plate—and by extension the end of the instrument—onto or into the pressure fluid passageway.

In assembled form as shown in FIG. 8A, FIG. 8B, and FIG. 9, the force plate bottom surface (520)—when the force plate (500) is present—is configured to interact with at least a portion of the top surface (230) of the pressure fluid passageway (200). Extending from a portion of the force plate bottom surface—preferably from an inner portion of the force plate bottom surface—may be a force plate lip (530). This force plate lip is configured to interact with a top surface of the instrument (20) when the instrument is connected to the instrument tee by the first clamp member (300) as shown in FIG. 9.

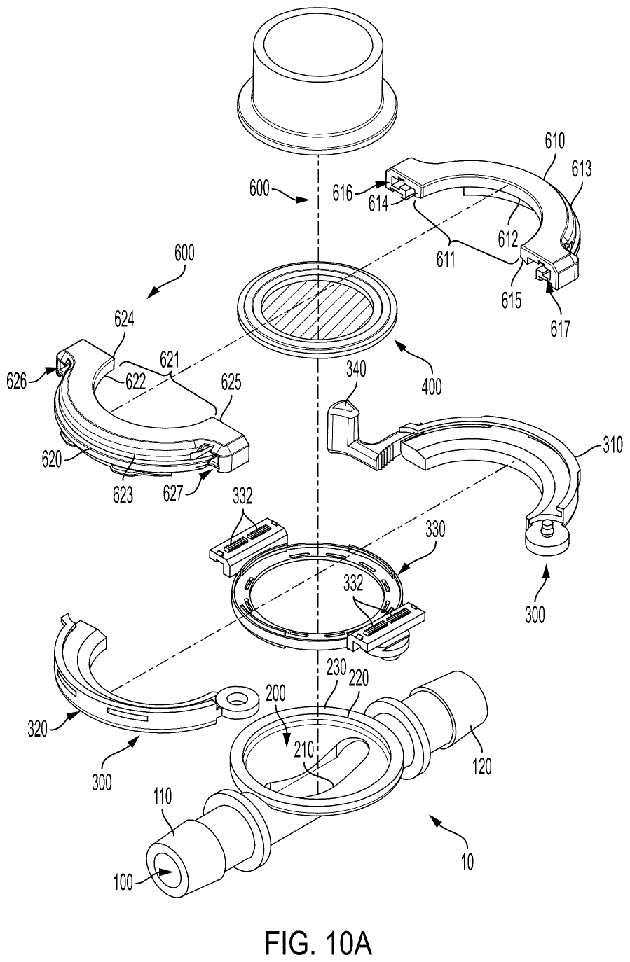

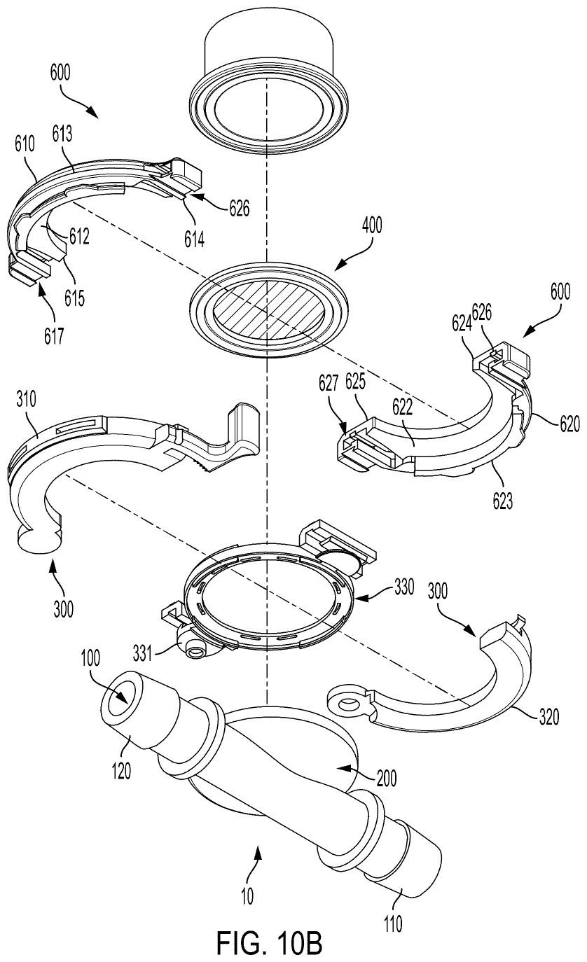

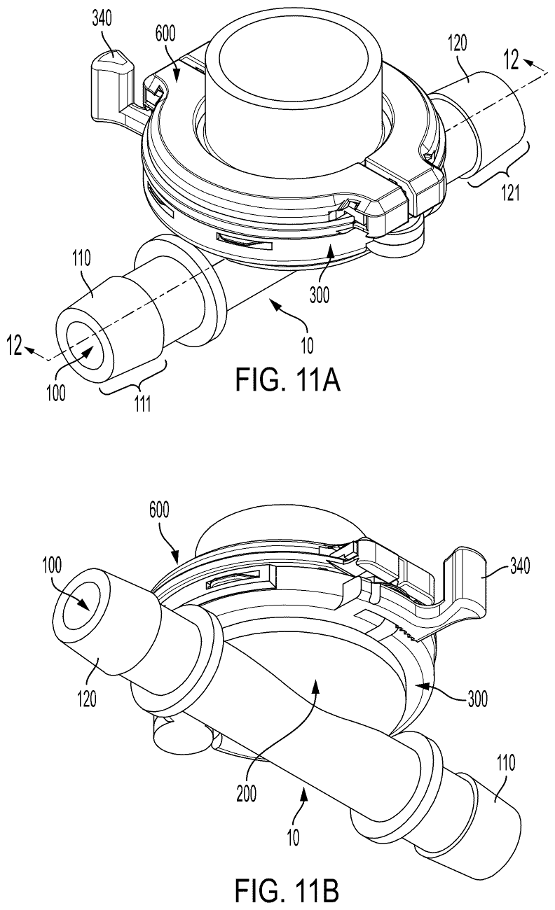

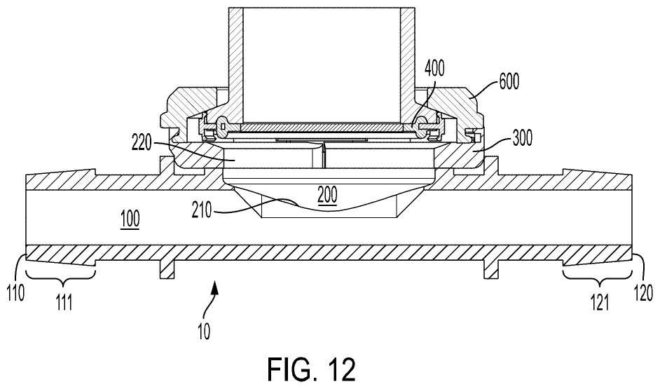

FIG. 10A through FIG. 12 shows an alternative embodiment in which the instrument tee (10) includes a second clamp member (600) with FIG. 10A and FIG. 10B showing an exploded view thereof, FIG. 11A and FIG. 11B showing an assembled view thereof, and FIG. 12 showing the embodiment in cross section. As shown in the Figures, the second clamp member may comprise a second clamp member first section (610) and a second clamp member second section (620). While the second clamp member may be utilized in embodiments where the first clamp member (300) is removably connected to the distal end (220) of the pressure fluid passageway (200), it is preferred for embodiments in which the first clamp member—including both the first clamp member first section (310) and the first clamp member second section (320)—is integrally connected to the distal end of the pressure fluid passageway.

As shown in FIG. 10A and FIG. 10B, the second clamp member first section (610) includes a third semi-circular wall (611). Attached to opposing end points (614 and 615 as shown in FIG. 10A) of the third semi-circular wall are a third wall first engagement member (616) and a third wall second engagement member (617). Similarly, the second clamp member second section (620) includes a fourth semi-circular wall (621). Attached to opposing end points (624 and 625) of the fourth semi-circular wall are a fourth wall first engagement member (626) and a fourth wall second engagement member (627). The third wall first engagement member may be configured to connect to the fourth wall first engagement member while the third wall second engagement member may be configured to connect to the fourth wall second engagement member.

The engagement members (616, 617, 626, and 627) of the second clamp section (600)—in some embodiments—may include a toothed tongue and groove engagement member in which a toothed tongue on the engagement member at one end point of one second clamp member section is configured to connect to a toothed groove on the engagement member at one end point of an opposing second clamp member section. Preferably, the teeth will be fine teeth defined by having an integral tooth dimension of at least 1.02 mm and no greater than 1.78 mm. The integral tooth dimension may also be at least 1.14 mm, or at least 1.27 mm. The integral tooth dimension may also be no greater than 1.65 mm, no greater than 1.52 mm, or no greater than 1.40 mm. The integral tooth dimension may also be defined as a range selected from the group consisting of between 1.02 mm and 1.65 mm, between 1.02 mm and 1.52 mm, between 1.02 mm and 1.40 mm, between 1.14 mm and 1.78 mm, between 1.14 mm and 1.65 mm, between 1.14 mm and 1.52 mm, between 1.14 mm and 1.40 mm, between 1.27 mm and 1.78 mm, between 1.27 mm and 1.65 mm, between 1.27 mm and 1.52 mm, and between 1.27 mm and 1.40 mm.

In some embodiments, the first clamp member (300) and second clamp member (600) may include a first joining member (330) as shown in FIG. 10A and FIG. 10B. When present, the first joining member may include a cylindrical section having a number of features—such as joining member teeth (332) and/or a hinge mount (331) which assist in attaching the first hinge section to the second hinge section—connected thereto to assist in securely connecting the first clamp member first section (310) to the first clamp member second section (320) and/or to assist in securely connecting the second clamp member first section (610) to the second clamp member second section (620).

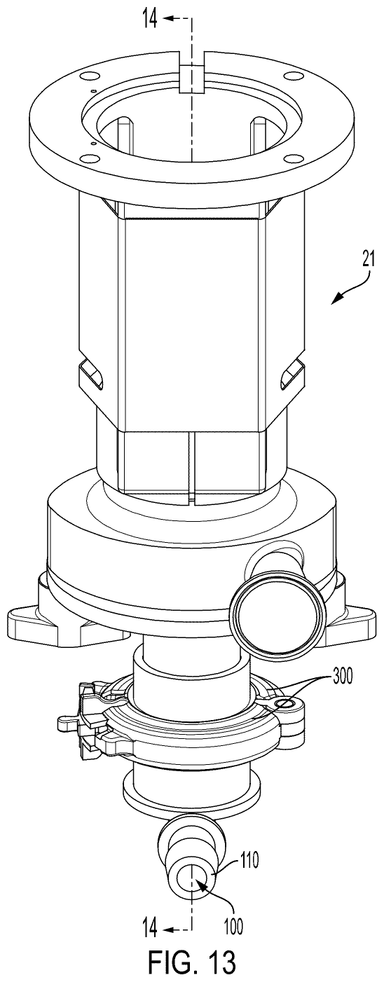

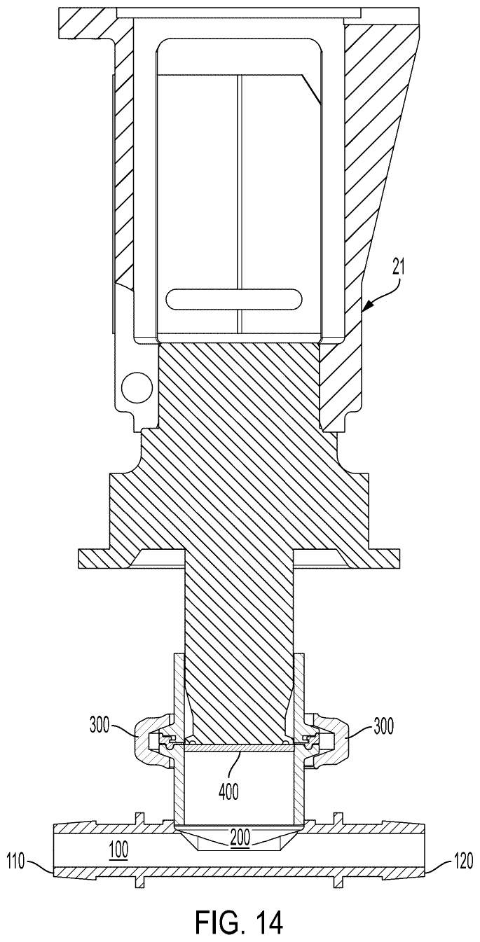

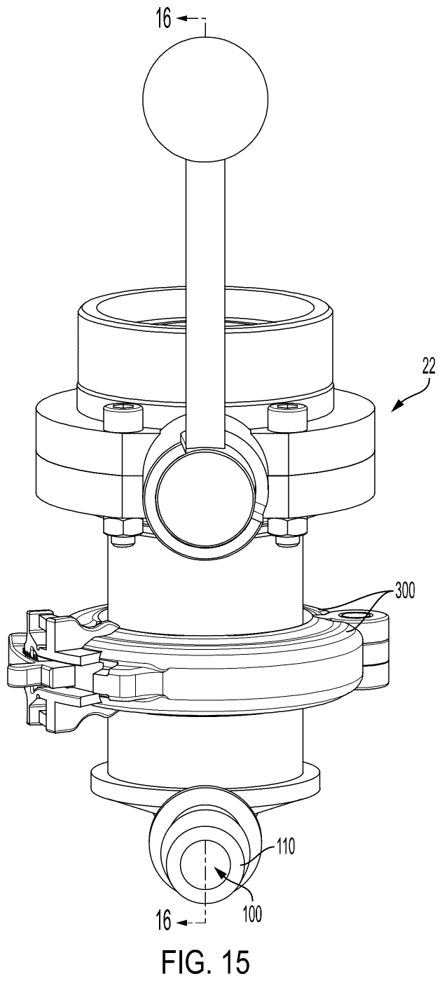

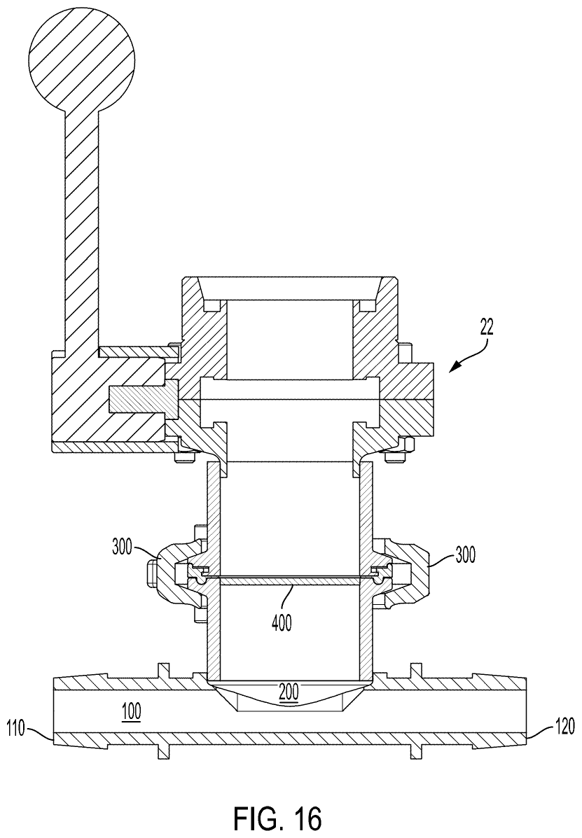

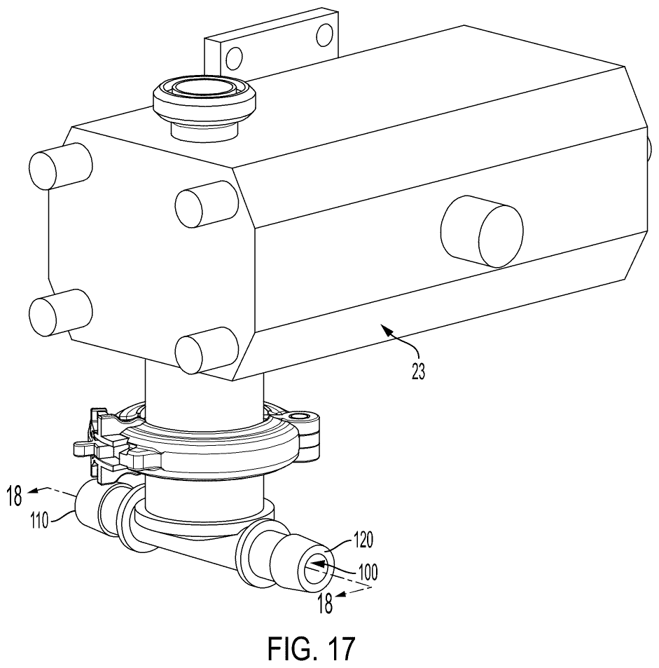

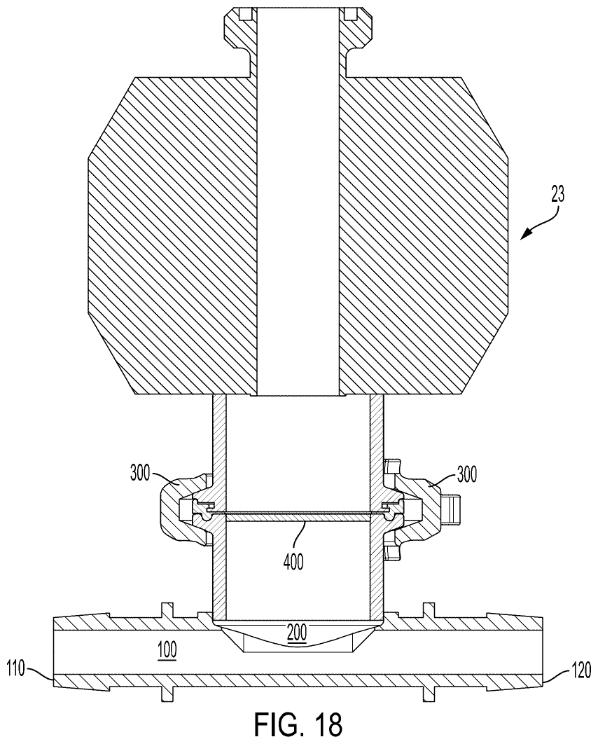

FIG. 13 to FIG. 18 show embodiments of the instrument tee (10) in which the first clamp member (300) is integrated into the end of the instrument (20 as shown in FIG. 1) to which the instrument tee is connected. FIG. 13 and FIG. 14 show the first clamp member integrated into the end of a centrifugal pump (21). FIG. 15 and FIG. 16 show the first clamp member integrated into the end of a valve (22). FIG. 16 and FIG. 17 show the first clamp member integrated into the end of a positive displacement pump (23). While the Figures show examples of integrated the end of a centrifugal pump, a valve, and a positive displacement pump—the invention is not so limited and other embodiments may exist. The first clamp member may therefore be integrated into the end of any instrument which may be connected to a pressure fluid passageway (200) of an instrument tee such as a pressure gauge, a temperature gauge, a pump, a valve, and the like.

Integrating the first clamp member (300) into the end of the instrument (20) may be accomplished in several ways. The simplest of which involves manufacturing the end of the instrument which is to be connected to the pressure fluid passageway (200) and the first clamp member of a single piece of material with a portion of the first inner surface (312 as shown in FIG. 4) extending from an outer surface of the end of the instrument. In other embodiments, the first clamp member and the end of the instrument may be manufactured separately, and a portion of the first inner surface may be permanently connected to the outer surface of the end of the instrument such as by welding or an adhesive bond.

The components of the instrument tee disclosed herein—including the main fluid passageway, the pressure fluid passageway, the first clamp member, the force plate, and/or the second clamp member—may be fabricated of any number of materials utilizing any number of manufacturing techniques. Non-limiting examples of such materials include polymeric materials, glass filled polymer materials—specifically glass filled nylon, and silicone materials. Non-limiting examples of such manufacturing techniques include injection molding and additive manufacturing (also referred to as 3D printing).

The presence of the gasket which fluidly isolates the pressure fluid passageway from the instrument reduces or eliminates the possibility that fluids—such as biopharmaceuticals—will be contaminated by metal instrument components during operation. Additionally, the relatively short, angled pressure fluid passageway allows for more accurate measurement of pressures within the main fluid passageway while reducing or eliminating dead zones.

While the instrument tee has been described as having one or more exemplary designs, the present instrument tee may be further modified within the spirit and scope of this disclosure. This application is therefore intended to cover any variations, uses, or adaptations of the system using its general principles.

What is claimed is:

1. An instrument tee (10) comprising:
a main fluid passageway (100) comprising a first fluid port (110), a second fluid port (120) opposite the first fluid port, and a pressure fluid passageway port (130) between the first fluid port and the second fluid port;
a pressure fluid passageway (200) comprising a proximal end (210) fluidly connected to the pressure fluid passageway port and having a first inner diameter (211) and a first outer diameter (212), and a distal end (220) having a second inner diameter (221) and a second outer diameter (222);
a first clamp member (300) configured to connect an instrument (20) to the distal end of the pressure fluid passageway; and
a gasket (400) a first surface (410) of which is configured to connect to at least a portion of a top surface (230) of the pressure fluid passageway, and a second surface (420) of which is configured to connect to a bottom surface of the instrument when the instrument is connected to the instrument tee by the first clamp member; and
wherein the first inner diameter is not the same as the second inner diameter.

2. The instrument tee of claim 1, wherein the first inner diameter is greater than the second inner diameter.

3. The instrument tee of claim 1, wherein the first inner diameter is less than the second inner diameter.

4. The instrument tee of claim 1, wherein the first fluid port comprises a first hose barb (111).

5. The instrument tee of claim 1, wherein the second fluid port comprises a second hose barb (121).

6. The instrument tee of claim 1, further comprising the instrument removably connected to the pressure fluid passageway by at least the first clamp member.

7. The instrument tee of claim 6, wherein the instrument is a pressure gauge.

8. The instrument tee of claim 7, wherein the first clamp member is integrally connected to an end of the pressure gauge.

9. The instrument tee of claim 6, wherein the instrument is a centrifugal pump.

10. The instrument tee of claim 9, wherein the first clamp member is integrally connected to an end of the centrifugal pump.

11. The instrument tee of claim 6, wherein the instrument is a valve.

12. The instrument tee of claim 11, wherein the first clamp member is integrally connected to an end of the valve.

13. The instrument tee of claim 6, wherein the instrument is a positive displacement pump.

14. The instrument tee of claim 13, wherein the first clamp member is integrally connected to an end of the positive displacement pump.

15. The instrument tee of claim 6, wherein the gasket is configured to fluidly isolate the pressure fluid passageway from the instrument.

16. The instrument tee of claim 1, wherein the first clamp member comprises:
a first clamp member first section (310) having:
a first semi-circular wall (311) having a first inner surface (312), a first outer surface (313), a first wall first end point (314), and a first wall second end point (315);
a first wall first engagement member (316) attached to the first wall outer surface at the first wall first end point and comprising a first hinge section (317); and
a first wall second engagement member (318) attached to the first outer surface at the first wall second end point and comprising a first plurality of inwardly facing teeth (319); and
a first clamp member second section (320) having:
a second semi-circular wall (321) having a second inner surface (322), a second outer surface (323), a second wall first end point (324), and a second wall second end point (325);
a second wall first engagement member (326) attached to the second outer surface at the second wall first end point and comprising a second hinge section (327); and
a second wall second engagement member (328) attached to the second outer surface at the second wall second end point and comprising a first plurality of outwardly facing teeth (329); and
wherein the first clamp member first section is configured to connect to the first clamp member second section by mating the first hinge section to the second hinge section and mating the first plurality of inwardly facing teeth to the first plurality of outwardly facing teeth.

17. The instrument tee of claim 1, further comprising a force plate (500) having a force plate top surface (510) a portion of which is configured to interact with the first clamp member, a force plate bottom surface (520) configured to interact with at least a portion of the top surface of the pressure fluid passage, and a force plate lip (530) extending from the force plate bottom surface and configured to interact with a top surface of the instrument when the instrument is connected to the instrument tee by the first clamp member.

18. The instrument tee of claim 1, further comprising a second clamp member (600) configured to connect the instrument (20) to the distal end of the pressure fluid passageway.

19. The instrument tee of claim 18, wherein the second clamp member is integrally connected to the distal end of the pressure fluid passageway.

20. The instrument tee of claim 18, wherein the second clamp member comprises:
a second clamp member first section (610) having:
a third semi-circular wall (611) having a third inner surface (612), a third outer surface (613), a third wall first end point (614), and a third wall second end point (615);
a third wall first engagement member (616) attached to the third outer surface at the third wall first end point; and
a third wall second engagement member (617) attached to the third outer surface at the third wall second end point; and
a second clamp member second section (620) having:
a fourth semi-circular wall (621) having a fourth inner surface (622), a fourth outer surface (623), a fourth wall first end point (624), and a fourth wall second end point (625);
a fourth wall first engagement member (626) attached to the fourth outer surface at the fourth wall first end point; and
a fourth wall second engagement member (627) attached to the fourth outer surface at the fourth wall second end point; and
wherein the third wall first engagement member is configured to connect to the fourth wall first engagement member, and the third wall second engagement member is configured to connect to the fourth wall second engagement member.
